(19) United States
(12) Patent Application Publication
(10) Pub. No.: US 2020/0199573 A1
(43) Pub. Date: Jun. 25, 2020

(54) Polynucleotide Purification Agents and Related Methods

(71) Applicant: ACTAScience, Inc., North Andover, MA (US)

(72) Inventors: JIN YUAN, N. Andover, MA (US); DAI SHI, Boxford, MA (US)

(21) Appl. No.: 16/725,659

(22) Filed: Dec. 23, 2019

Related U.S. Application Data
(60) Provisional application No. 62/784,142, filed on Dec. 21, 2018.

(51) Int. Cl. C12N 15/10

(52) U.S. Cl. CPC C12N 15/1006

(57) ABSTRACT

The invention purifies desired polynucleotide samples by removing impurities resulting from biochemical reactions or physical manipulation, such as excess oligonucleotides, salt and protein after a polymerase chain reaction (PCR), or by selecting polynucleotide of desired sizes from a mixture of polynucleotides of different sizes resulting from polynucleotide fragmentation procedures. Desired polynucleotides bind reversibly to a solid surface such as magnetic micro-particles whose surfaces are coated with or without a functional group, such as a carboxyl group, during the removal process of impurity or polynucleotides of unintended sizes. The polynucleotides can be DNA, RNA or polyamide nucleic acids (PNAs). As a result, polynucleotides bound to the solid surface are purified or selected for desired sizes.

The present invention utilizes polyalkylene glycol of certain concentrations as removal method of impurity or polynucleotide of un-desirable sizes instead of using ethanol or isopropanol of certain concentrations, such as 70% or 80%, which have been used as standard method of impurity removal for the past 20 years.

### CROSS-REFERENCE TO RELATED APPLICATIONS

This application claims priority to and the benefit of co-pending U.S. provisional patent application Ser. No. 62/784,142, filed Dec. 21, 2018, which application is incorporated herein by reference in its entirety.

### FIELD OF THE INVENTION

The invention relates to method of nucleotide and polynucleotide isolation, purification, and size selection methods in general and particularly to agents and protocol that relies on a wash solution based on one or more polyalkylene glycols (PAGs) such as polyethylene glycol (PEG).

### BACKGROUND OF THE INVENTION

In molecular biology and biochemistry, being able to prepare, purify and handle high quality polynucleotides (e.g., DNA, RNA, PNA) is critical to success and indispensable to quality control. Binding polynucleotides to a solid surface such as magnetic microparticles is a widely used strategy to separate polynucleotides from impurities, which can be a multitude of items: inorganic contaminants, enzymes, cellular debris, oligonucleotide fragments, salts and so on. It is also often required to select polynucleotides of certain sizes from a mixture of polynucleotides of different sizes in many molecular biology applications, such as Next-Generation Sequencing. After discarding the supernatant containing majority amount of impurities and polynucleotides of un-intended sizes, washing the remaining polynucleotides bound to the solid surface with a wash solution is critical to remove residual amounts of both impurities and polynucleotide of unintended sizes, both of which might otherwise negatively impact downstream biochemical reactions. Meanwhile, it is critical that the bound polynucleotides are not lost from the solid surface during this removal of impurities and/or polynucleotides of unintended sizes.

For the last 20 plus years, using a low molecular weight alcohol-based solution, e.g., ethanol or isopropanol of certain concentrations, such as 70% or 80%, has been the standard protocol for removing impurities or polynucleotides of unintended sizes from polynucleotides bound to a solid surface.

However, ethanol or isopropanol based removal protocols have multiple undesirable effects: first, residual low molecular weight alcohol such as ethanol or isopropanol left after removal often has negative impacts on downstream biomedical reactions such as sequencing. As a result, certain amount of waiting time is usually required for residual ethanol or isopropanol to evaporate to minimize such risks, which slows down the entire purification or size selection process. While non-completion of evaporation of residual ethanol or isopropanol (also called under-drying) negatively affects downstream biochemical reactions, over-drying of solid surfaces such as magnetic microparticles tends to cause difficulties in eluting bound polynucleotides into a liquid solution at the end of the purification process, which reduces the yield of the polynucleotides. And because of concerns of under-drying and over-drying, time and effort is required from the operator to frequently check during the low-molecular-weight-alcohol evaporation period. Since under- or over-drying, in most cases, is solely judged through visual inspection, a process that is highly subjective to human error and variation, the use and subsequent removal of low-weight alcohol from the samples tend to negatively affect the reproducibility of the laboratory result.

In addition, due to high volatility of ethanol or isopropanol, frequent preparation, e.g. daily preparation, of fresh 70% or 80% ethanol or isopropanol is required in most of low molecular weight alcohol-based impurity removal or size selection protocols. This adds a substantial burden to the laboratory staff

There is a need for simpler, faster and more reliable ways to purify or size select polynucleotides.

### SUMMARY OF THE INVENTION

The present invention provides a novel way of purifying or size selecting polynucleotides that is simpler and faster than currently standard protocols. By using a wash solution that does not include any significant amount, i.e., no more than 0.01% by volume, of low-molecular-weight alcohol, the present invention discloses a method for the removal of impurities, or polynucleotides of unintended sizes, from polynucleotides that are reversibly and preferably, non-specifically at least in part, bound to an anchoring surface, preferably a solid surface, such as that of a magnetic microparticle. The method results in the removal of impurities such as excess oligonucleotides, salt and protein, or polynucleotides of unintended sizes, while preventing polynucleotide loss from the bound surface. And with the elimination of evaporative, low-weight alcohol use from the method of the invention, all the undesired effects and shortcomings described above for the conventional polynucleotide purification or size-selection protocol are therefore avoided: no more need to prepare fresh 70% or 80% ethanol or isopropanol solutions before each laboratory session, no more waiting time or visual inspection and/or subjective judgment for drying the low-weight alcohol in the samples in every purification or size-selection operation, and so on.

In one aspect, the invention relates to a method using, as a wash solution or buffer for polynucleotide purification or size selection, an aqueous solution containing polyalkylene glycol (PAG), which can be, e.g., polyethylene glycol (PEG), polypropylene glycol (PPG) or a mixture of both, provided that no low molecular weight alcohol is used in the wash. The method enables the user to obviate the need to prepare and use prior alcohol-based wash solutions.

In one embodiment, the invention provides a method of purifying polynucleotides without the use of any low molecular weight alcohol-based wash, the method comprising the sequential steps of:
a. contacting a surface with a solution containing polynucleotides and impurities;
b. allowing binding between the surface and the polynucleotides to take place;
c. washing the polynucleotides-bound surface with a wash solution comprising essentially of polyalkylene glycol and water, optionally with 1-30 mM ionic salt, e.g., 5-10 mM magnesium ions (inclusive of both ends), to remove impurities on the surface; and, preferably,
d. eluting the polynucleotides from the surface.

In another embodiment, the invention provides a method of selecting polynucleotides of certain sizes without the use of any low molecular weight alcohol-based wash, the method comprising the sequential steps of:
a. contacting a surface with a solution containing a mixture of polynucleotides of different sizes including both desired and unintended sizes;
b. allowing binding between the surface and the polynucleotides of certain sizes to take place;
c. washing the polynucleotides-bound surface with a wash solution comprising essentially of polyalkylene glycol and water, optionally with 1-30 mM ionic salt, e.g., 5-10 mM magnesium ions (inclusive of both ends), to remove unbound polynucleotides of unintended sizes on the surface; and, preferably,
d. eluting the bound polynucleotides of desired sizes from the surface.

In another aspect, the invention features a purification kit for polynucleotides. The kit includes a solid phase binding surface and a binding buffer for facilitating reversibly binding polynucleotides of interest in a solution to the solid phase binding surface, and a wash solution comprising essentially of polyalkylene glycol and water, optionally with 1-30 mM ionic salt, e.g., 5-10 mM magnesium ions (inclusive of both ends), for removing impurities from the binding surface while retaining most of the bound polynucleotides of interest. The solid phase binding surface can be any of the support or substrate surface used in conventional nucleotide purification procedures, for example, (magnetic) microparticles or beads, (silica) resins, membranes, filters, fibers, (silica) matrices, and so on. In an embodiment, the kit includes magnetic microparticles or nanoparticles for providing such binding surfaces. In another embodiment, the binding buffer includes a suitable salt and a polyalkylene glycol at concentrations suitable for reversibly binding polynucleotides onto the solid phase surfaces, such as to the surfaces of magnetic microparticles. The kit may additionally comprise a suitable elution buffer, reagents for preparing such buffers, or reagents for preparing a lysate, e.g., a clear lysate. In an embodiment, the elution buffer consists essentially of water.

In another aspect, the invention features a size selection kit for polynucleotides e.g., a DNA library. The kit includes a solid phase binding surface and a binding buffer for facilitating reversibly binding polynucleotide of intended sizes in a solution to the solid phase binding surface, and a wash solution comprising essentially of polyalkylene glycol and water, optionally with 1-30 mM ionic salt, e.g., 5-10 mM magnesium ions (inclusive of both ends), for removing unbound polynucleotides of un-intended sizes from the binding surface while retaining most of the bound polynucleotide of interest. The solid phase binding surface can be any of the support or substrate surface used in conventional nucleotide purification procedures, for example, (magnetic) microparticles or beads, (silica) resins, membranes, filters, fibers, (silica) matrices, and so on. In an embodiment, the kit includes magnetic microparticles or nanoparticles for providing such binding surfaces. In another embodiment, the binding buffer includes a suitable salt and a polyalkylene glycol at concentrations suitable for reversibly binding polynucleotides of certain sizes onto the solid phase surfaces, such as to the surfaces of magnetic microparticles. The kit may additionally comprise a suitable elution buffer, reagents for preparing such buffers, or reagents for preparing a lysate, e.g., a clear lysate. In an embodiment, the elution buffer consists essentially of water.

The method of the present invention has applicability in essentially any context in which separation polynucleotide from the rest of the sample or size selection of polynucleotides is desired. In addition, method of the present invention greatly simplifies, speeds up, and standardizes the manipulations carried out involving polynucleotides. For example, the present method simplifies the purification of PCR products, or the isolation of cloned DNA from lysate, or size selection of a DNA library, by obviating the need for preparing conventional alcohol-based wash solutions or the subsequent time and uncertainty associated with drying the low-weight alcohol content, and produces polynucleotides ready for sequencing and further characterization and processing. The present method and kit also have the advantage of lowering the cost and making it simpler to perform and produce high-quality polynucleotides in high yields. These properties, coupled with its applicability to many procedures useful in molecular biology, make the method of the present invention amenable to automation and high throughput in isolating polynucleotides a commercial possibility, e.g., with the 96-well format.

The foregoing and other objects, aspects, features, and advantages of the invention will become more apparent from the following description and from the claims.

### BRIEF DESCRIPTION OF THE DRAWINGS

The objects and features of the invention can be better understood with reference to the drawings described below, and the claims. The drawings are not necessarily to scale, emphasis instead generally being placed upon illustrating the principles of the invention. In the drawings, like numerals are used to indicate like parts throughout the various views.

FIG. 1 illustrates exemplary embodiments of using a wash solution according to the invention to successfully purify DNA samples (removal of oligo polynucleotides of 50 bp), where an agarose gel image shows that DNA fragment sizes and concentrations from samples purified with wash solution containing Polyethylene Glycol (Lanes F1, G1, and H1) are equal to or greater than those purified with 70% ethanol (Lane E1), and that wash solution containing Polyethylene Glycol completely removed oligo-/poly-nucleotides of 50 bp as intended. Lane A1 is the DNA fragment size marker, and “bp” stands for “base pair.”

FIG. 2 compares an exemplary embodiment using a wash solution to purify DNA samples without air-drying before undergoing successful PCR amplification according to the invention. Specifically, 10 μL DNA solution was used for real-time PCR after a purification process using inventive wash solution containing Polyethylene Glycol (without any air-drying), or with a traditional 70% ethanol (with air-drying steps).

FIG. 3 illustrates exemplary embodiment using a wash solution according to the invention for the successful removal of impurity EDTA, which inhibits PCR, from DNA samples before undergoing PCR amplification: 10 μL DNA/EDTA solution was used for real-time PCR amplification with or without purification with wash solution containing Polyethylene Glycol. The horizontal line at “0” value of RFU is from unpurified DNA. The “S” shape curve rising from 0 to nearly 7000RFU at cycle 40 is from purified DNA with the wash solution.

FIG. 4 illustrates side-by-side comparison that shows DNA recovered from wash solution containing Polyethylene Glycol (right-hand bar) is greater than that from 70% ethanol (left-hand bar). Variation of result between replicates from wash solution containing Polyethylene Glycol (±3%) is much smaller than 70% ethanol (±23%).

FIG. 5 illustrates successful applications of RNA samples purified according to principles of the invention in downstream biochemical reactions: 10 μL RNA solution was used for reverse transcription/real-time PCR reaction after purification with wash solution containing Polyethylene Glycol or with 70% ethanol. Results indicate that samples processed with wash solution containing Polyethylene Glycol according to an inventive embodiment yielded highly equivalent amplification results that are highly comparable to those with 70% ethanol.

FIG. 6 illustrates exemplary embodiment using a wash solution according to the invention and positive impact of Magnesium in the wash solution. Specifically, a 2% agarose gel image shows 100 ng DNA fragment mixture of 1000, 900, 800, 700, 600, 500, 400, 300, 250, 200, 150, 100, and 50 base pair (top to bottom) after electrophoresis: sample without undergoing purification was used as negative control and DNA marker (Left Lane); DNA purified according to principles of the invention using wash solution containing Polyethylene Glycol with Magnesium (Middle Lane), or by wash solution containing Polyethylene Glycol without Magnesium (Right Lane), was assessed side by side.

FIG. 7 is a 2% agarose gel image showing 100 ng DNA fragment mixture of 1000, 900, 800, 700, 600, 500, 400, 300, 250, 200, 150, 100, 50 base pair (top to bottom) after electrophoresis: sample without purification or purified with six different wash solutions: positive control and DNA fragment size marker (Lane 1); Polyethylene Glycol at a concentration of 20-50% range (w/v) with Magnesium Chloride at a concentration of 1-30 mM range (Lane 2); 70% ethanol (Lane 3); 1% low molecular weight Polyethylene Glycol (v/v, molecular weight less than 200) with Magnesium Chloride (Lane 4); 1% medium molecular weight Polyethylene Glycol (w/v, molecular weight between 200 and 1000) with Magnesium Chloride (Lane 5); 1% high molecular weight Polyethylene Glycol (w/v, molecular weight between 1000 and 10000) with Magnesium Chloride (Lane 6); 1% very high molecular weight Polyethylene Glycol (w/v, molecular weight higher than 10000) with Magnesium Chloride (Lane 7).

FIG. 8 is a TapeStation 2200 (Agilent) analysis image showing size selection of 150 ng DNA fragment mixture of 1000, 900, 800, 700, 600, 500, 400, 300, 250, 200, 150, 100, 50 base pairs, respectively (top to bottom) after electrophoresis: Negative Control (without size selection) as DNA fragment size marker (left lane); DNA after size selection with a wash solution comprising Polyethylene Glycol at a concentration of 20-50% range (w/v) and Magnesium Chloride at a concentration of 1-30 mM range (right lane); and 80% ethanol (middle lane).

### DETAILED DESCRIPTION OF THE INVENTION

### I. Definition

Unless otherwise noted, technical terms are used according to conventional usage. Definitions of common terms in molecular biology may be found, for example, in J. Krebs et al. (eds.), Lewin's Genes XII, published by Jones and Bartlett Learning, 2017 (ISBN 9781284104493); Robert A. Meyers (ed.), Molecular Biology and Biotechnology: a Comprehensive Desk Reference, published by Anmol Publications Pvt. Ltd, 2011 (ISBN 9788126531783); and other similar technical references.

As used in the specification and claims, the singular form “a”, “an”, or “the” includes plural references unless the context clearly dictates otherwise. For example, the term “a cell” includes a plurality of cells including mixtures thereof. It is further noted that the claims may be drafted to exclude any optional element. As such, this statement is intended to serve as support for the recitation in the claims of such exclusive terminology as “solely,” “only” and the like in connection with the recitation of claim elements, or use of a “negative” limitations, such as “wherein [a particular feature or element] is absent,” or “except for [a particular feature or element],” or “wherein [a particular feature or element] is not present (included, etc.) . . . ”

As used herein, the recitation of a numerical range for a variable is intended to convey that the invention may be practiced with the variable equal to any of the values within that range. Thus, for a variable that is inherently discrete, the variable can be equal to any integer value within the numerical range, including the end-points of the range. Similarly, for a variable that is inherently continuous, the variable can be equal to any real value within the numerical range, including the end-points of the range. As an example, and without limitation, a variable which is described as having values between 0 and 2 can take the values 0, 1 or 2 if the variable is inherently discrete, and can take the values 0.0, 0.1, 0.01, 0.001, or any other real values >0 and <2 if the variable is inherently continuous.

As used herein, the term “about” means within plus or minus 10%. For example, “about 1” means “0.9 to 1.1”, “about 2%” means “1.8% to 2.2%”, “about 2% to 3%” means “1.8% to 3.3%”, and “about 3% to about 4%” means “2.7% to 4.4%.”

The method of the invention is useful for purifying, isolating, increasing the concentration, and size selecting double or single stranded polynucleotides of interest, whether or not they are further conjugated or formed within a complex, of virtually any size and molecular weight, and from virtually any sources. In a non-limiting embodiment, the present invention may be practiced to purify polynucleotides no shorter than about 10, 20, 30, 40 bp, or more, preferably no shorter than about 50 bp. The present method can be used, for example, to purify or separate polynucleotides present in a mixed solution containing both polynucleotides of interest and impurities. Examples of such a mixed solution include sample solutions containing cellular contents such as lysate from transfected host cells, DNA resulting from an amplification process (e.g., polymerase chain reaction (PCR)) and polynucleotides-containing semi-solids such as gels including agarose gel samples. In addition, the present method can be used to separate and remove impurities from polynucleotides bound to a surface, preferably a solid phase surface such as (magnetic) microparticles or beads, (silica) resins, membranes, filters, fibers, (silica) matrices, ion-exchange chromatography columns, and so on. The present method can also be used to remove polynucleotide of unintended sizes from polynucleotides of desired size(s), e.g. a DNA library bound to a surface, preferably a solid phase surface such as (magnetic) microparticles or beads, (silica) resins, membranes, filters, fibers, (silica) matrices, ion-exchange chromatography columns, and so on.

After purification and/or size selection, polynucleotides such as DNA, RNA or PNA bound to the solid surface can be eluted into aqueous solution and be used for downstream applications, such as Sanger sequencing and Next-generation sequencing. Because the method of the present invention is useful with both single and double stranded polynucleotides, as well as a wide range of polynucleotide fragment sizes, it has applicability in essentially any context in which polynucleotide purification or size selection is desired. In addition, this method permits standardization and automation of the purification step in molecular biology and biochemistry.

The present invention greatly simplifies the purification or size selection process and yields purified polynucleotides of high quantity and quality. Compared to standard ethanol- or isopropanol-based impurity removal or size selection procedures, the present invention offers at least the following advantages: (1) residual polyalkylene glycol used as the main/exclusive ingredient of the wash solution after removal of impurities or polynucleotide of unintended sizes has no impact on downstream biochemical reactions, which improves the performance and reproducibility of such downstream biochemical reactions; (2) because of (1), the present invention eliminates waiting time required previously for evaporation of residual low-weight alcohol, e.g., ethanol (C₂H₆O) or isopropanol (C₃H₈O)—this enables a quicker purification or size selection process and higher throughput; (3) because of (1), the present invention eliminates the need for visually checking for the completion of alcohol evaporation and possible over-drying of the solid surfaces, e.g., of microparticles; (4) because of (3), the invention saves time and effort for the operator; (5) because of (3), subjectivity and human error in judging completion of alcohol evaporation or possible over-drying of the solid surface are eliminated, which improves reproducibility of the downstream operations; and (6) the present invention eliminates time and effort for frequent preparation of fresh 70% or 80% ethanol or isopropanol that is required in most of the conventional impurity removal or size selection methods that rely on the use of low-weight alcohols.

The term “low-weight alcohol,” as used herein, refers to alcohols with molecular weight less than 100, which includes, e.g., primary alcohols such as ethanol and isopropanol.

As used herein, the terms “polynucleotide,” “oligonucleotide” and “nucleic acid” are used interchangeably throughout and include DNA molecules (e.g., cDNA, genomic DNA, chromosomal DNA, and plasmid DNA), RNA molecules (e.g., mRNA), analogs of the DNA or RNA generated using nucleotide analogs (e.g., peptide nucleic acids (PNA) and non-naturally occurring nucleotide analogs), and hybrids thereof. The nucleic acid molecule can be single-stranded or double-stranded. The double-stranded nucleic acid may have the two strands chemically linked and/or form at least one double-stranded region under suitable annealing conditions. The double-stranded region may contain at least one gap, nick, bulge, and/or bubble.

The surface used in the present invention to reversibly bind the polynucleotides of interest is preferably solid, but can be semi-solid such as gelatin-like substrates. The surface can be coated with silica or otherwise equipped with functional groups that aid in attracting and (temporarily) anchoring, during the purification or size-selection procedure, polynucleotides of interest including those of desired lengths.

For example, in the case of microparticle as a functional group-coated surface to practice the invention, in an embodiment, the surface of the microparticles is coated with moieties that each have a free functional group bound to the amino group of the amino silane on the microparticle. The functional group acts as a bio-affinity absorbent for the polynucleotides, e.g., DNA, in the sample solution. In one embodiment, the functional group is a carboxylic acid. A suitable moiety with a free carboxylic acid functional group is a succinic acid moiety in which one of the carboxylic acid groups is bonded to the amine of amino silanes through an amide bond and the second carboxylic acid is unbonded, resulting in a free carboxylic acid group attached or tethered to the surface of the microparticle, e.g., magnetic ones. Carboxylic acid-coated magnetic microparticles are commercially available from GE Healthcare. Other suitable functional groups that can be used for coating the surface of the microparticles include, but are not limited to, thiol groups (microparticles with thiol group coating are commercially available from PerSeptive Diagnostics, Division of PerSeptive Biosystems, Catalog Number 8-4135), streptavidin (microparticles with a streptavidin coating are commercially available from PerSeptive Diagnostics, BioMag Steptavidin, Catalog Number 8-MB4804), and amine groups (microparticles with a amine group coating are commercially available from Polysciences Catalog Number 07763-5).

In a preferred embodiment, magnetic microparticles are used to practice the present invention. As used herein, “magnetic microparticles” are microparticles attracted by a magnetic field. The magnetic microparticles used in the method of the present invention typically include a magnetic metal oxide core, which is generally surrounded by an absorptively or covalently bound silane coat to which a wide variety of bio-affinity adsorbents can be covalently bound through selected coupling chemistries, thereby coating the surface of the microparticles with functional groups. The magnetic metal oxide core is preferably iron oxide, wherein iron is a mixture of Fe²⁺ and Fe³⁺. The preferred Fe²⁺ versus Fe³⁺ ratio is about 2:1, but can vary from about 0.5:1 to about 4:1.

Suitable amino silanes useful for coating the microparticle surfaces include p-aminopropyltrimethoxysilane, N-2-aminoethyl-3-aminopropyltrimethoxysilane, triaminofunctional silane (H₂NCH₂—NH—CH₂CH₂—NH—CH₂—Si—(OCH₃)₃, n-dodecyltriethoxysilane and n-hexyltrimethoxysilane. Methods of preparing these microparticles are described in U.S. Pat. Nos. 4,628,037, 4,554,088, 4,672,040, 4,695,393 and 4,698,302, the teachings of which are hereby incorporated by reference into this application in their entirety. These patents disclose other amino silanes which are suitable to coat the iron oxide core and which are encompassed by this invention. Magnetic microparticles comprising an iron oxide core, as described above, without a silane coat (BioMag Iron Oxide particles available from PerSeptive Diagnostics, Division of PerSeptive Biosystems, Catalog Number 8-4200) can also be used in the method of the present invention.

Magnetic microparticles used in the present invention should be of such a size that their separation from solution, for example by filtration or magnetic separation, is not difficult. Suitable sizes range from about 0.1 μm in diameter to about 200 μm in diameter. Suitable magnetic microparticles are commercially available, e.g., from GE Healthcare and other vendors.

As used herein, “non-specific binding” or “binding non-specifically” refers to binding of different polynucleotide molecules with approximately the same affinity to support surfaces, e.g., of magnetic microparticles, despite differences in the nucleic acid sequence or size of the different polynucleotide molecules. As used herein, “reversible binding” or “binding reversibly” refers to non-covalent binding of different polynucleotide molecules to support surfaces, e.g., of magnetic microparticles, that can be undone under different conditions, e.g., salt ion concentrations, pH, or temperature in a surrounding solution.

The mixed solution containing polynucleotides of interest where the method of the present invention is typically practiced can contain DNA that is the reaction product of PCR amplification, or DNA/RNA produced from other enzymatic reactions, e.g. nucleic acid ligation reaction, primer extension reaction, reverse transcription, DNA fragmentation reaction, etc. The mixed solution can also be a lysate. A “lysate,” as used herein, is a solution containing cells that contain cloned DNA and/or genomic DNA and/or RNA and whose cellular membranes have been disrupted, with the result being that contents of the cell, including the DNA or RNA contained therein, are in the solution. One can further add RNase or DNase to create a lysate free of RNA or DNA, thereby allowing DNA to bind to the support surface, e.g., magnetic microparticles, free from RNA, or vise versa. Methods of creating a lysate are well known in the art, see Birnboim and Doly, Nucl. Acids Res., 7:1513 (1979), and Horowicz and Burke, Nucleic Acids Research 9:2989 (1981), the teachings of which are hereby incorporated by reference in their entirety. For example, a lysate can be produced by treating host cells with sodium hydroxide or its equivalent (0.2N) and sodium dodecyl sulfate (SDS) (1%).

Lysates coming from host cells such as bacterial cells, mammalian cells or yeast cells may contain exogenous or foreign DNA, in addition to their own genomic DNA. The foreign DNA may be introduced directly into the host cell by means known to one of ordinary skill in the art, e.g., bacterial artificial chromosomes (BAC), yeast artificial chromosomes (YAC), plasmids, cosmids and bacteriophage. In an embodiment, plasmid DNA is introduced into the host cell by a phage into which the plasmid DNA has been packaged. Host cells containing foreign DNA introduced by any method are herein referred to as “transfected host cells.”

The polynucleotides-containing mixed solution from which purification is conducted may also be an agarose solution. For example, a mixture of DNA is separated, according to methods known to one skilled in the art, e.g., by electrophoresis on an agarose gel. A plug of agarose containing the DNA of interest can be excised from the gel and added to 1-10 volumes of 0.5×SSC (0.75M NaCl, 0.0075M Sodium Citrate, pH 7.0). The mixture is then melted at a temperature from about 60° C. to about 100° C. for about one to about twenty minutes, preferably about ten minutes, to create an agarose solution containing the DNA of interest along with impurities.

The polynucleotide-containing mixed solution from which size selection is conducted may be a solution of mixed polynucleotide of different sizes. For example, a mixture of DNA of different sizes is generated, according to methods known to one skilled in the art, e.g., by enzymatic fragmentation using an enzyme reaction at 37° C. incubation for 5-30 minutes. The reaction is then stopped by high concentration, e.g., about 0.5M, of EDTA, to create a solution containing the DNA of desired sizes along with unintended sizes.

According to method of the present invention, after the mixed solution containing both polynucleotides of interest and impurities, or a mixture of polynucleotides of different sizes, contacts the anchoring surface, e.g., magnetic microparticles coated with a free functional group, reversible binding between the surface and polynucleotides of interest is allowed and encouraged to take place under suitable conditions. Such binding may be non-specific; and similar binding likely take place between the impurities and the anchoring surface as well. Suitable conditions for binding may include suitable salt concentrations and/or suitable polyalkylene glycol (PAG) (e.g., PEG) concentration in the solution. In an embodiment, a binding solution/buffer with a final salt (e.g., sodium chloride) concentration ranging from about 0.5M to about 5.0M and a final PEG concentration ranging from about 5% to about 20% may be introduced in order to achieve or encourage reversible binding between the polynucleotide of interest (e.g., DNA) in the solution and the anchoring surface, e.g., of a magnetic microparticle.

After binding has taken place (e.g., 1-30 minutes), the anchoring surface (e.g., microparticles) is separated from the rest of the mixed solution through the application of a magnetic force, a centrifugal force, and/or gravity or filtration as well known to one skilled in the art, the wash solution/buffer of the invention is introduced to remove impurities or polynucleotide of unintended sizes from the anchoring surface. Substances thus removed may include excess organic and inorganic matters, polynucleotide fragments of un-intended sizes including polynucleotides shorter than a certain size that are likely incomplete PCR products, primers, and/or salt molecules in excess of a suitable range (e.g. 1 mM or higher concentration of EDTA) for a downstream process such as PCR amplification.

The wash solution of the present invention is free of low-weight alcohols, and consists essentially of polyalkylene glycol (PAG) and water, that is, while the solution may include additional matters such as salt(s) besides these two components, they do not materially alter the functional capability of the wash solution to remove proportionally more impurities than polynucleotides of interest; preferably, the additional matters, in aggregate, do not occupy more than about 20% (w/v) or about 20% (v/v), or more preferably, not more than 3% (w/v) or 5% (v/v), depending on whether the additional matters are mostly solid or liquid at room temperature and under one atmosphere of pressure. In a non-limiting embodiment, the wash solution of the invention consists exclusively of PAG and water. PAGs are polymeric compounds with two hydroxyl (—OH) groups attached to two different carbon atoms in the polymeric chain. Examples of PAGs useful for practicing the method of the invention include and are not limited to: polyethylene glycol (PEG), polypropylene glycol (PPG), or mixtures of both. In a preferred embodiment, the PAG used is a PEG, commonly expressed as H—(O—CH₂—CH₂)ₙ—OH, also known as polyethylene oxide. PEGs can be prepared by polymerization of ethylene oxide and are commercially available over a wide range of molecular weights. For the present invention, the molecular weight of the polyethylene glycol (PEG) can range from about 100 to about 20,000, with a molecular weight of about 200-10,000 preferred. In an embodiment, the PEGs used has a molecular weight above 10,000. The concentration of PEG can be from 1% to 100%, and is preferably adjusted to about 20-50%, and most preferably to about 30% (v/v or w/v) in the wash solution of the present invention.

Salts that can be used in the wash solution of the invention include but are not limited to: sodium chloride (NaCl), lithium chloride (LiCl), potassium chloride (KCl), calcium chloride (CaCl₂), and magnesium chloride (MgCl₂). In an embodiment, magnesium chloride is used. In some embodiments, the ionic strength of the wash solution needs to be maintained high enough to keep polynucleotides of interest bound to the anchoring surface. Accordingly, the salt concentration in the wash solution is preferably adjusted to between about 0.001 mM and 1000 mM, and more preferably about 1-30 mM.

Other optional additives to the wash solution of the invention include but are not limited to: sodium azide, Proclin® and so on.

More than one wash solution can be used to wash impurities or polynucleotides of unintended sizes off the anchoring surface bound with polynucleotides of interest. The wash solution of the same formula can also be used on the same surface multiple times. After wash is completed, because PAG is often compatible with downstream processes, the sample may be ready for the next step without air-drying steps. In an embodiment, after wash, supernatant is removed, leaving the anchoring surface bound with polynucleotides of interest, and an elution buffer, e.g., water, is added to elute or unbind the polynucleotides from the surface. An elution buffer is any suitable solution that enables the polynucleotides of interest to unbind from an anchoring surface. In some cases, the elution buffer has a PAG concentration that is below the range required for the binding, and if the wash solution used contains salt, optionally, a salt concentration that is also below the range required for the binding of the polynucleotides to the surface and one that will not negatively impact the down stream procedures. Once the polynucleotides of interest are eluted, substrates with the anchoring surface (e.g., the microparticles) are separated from the elute, e.g., through application of a magnetic force, a centrifugal force, and/or gravity or filtration as well known to one skilled in the art. The polynucleotides of interests in the eluant are then further processed, e.g., undergo another biochemical reaction, in manners known to one skilled in the art.

According to one aspect of the invention, a purification kit is also provided which contains the reagents necessary for separating polynucleotides, such as DNA, RNA and PNAs, from a solution containing both polynucleotides of interest and impurities. The kit includes a solid phase binding surface, e.g., magnetic microparticles with a carboxyl group-coated surface, and a binding buffer for facilitating reversible binding. The solid phase binding surface, in various embodiments, can be selected from: a microparticle, a beads, a membrane, a filter, a fiber, and a matrix. The binding buffer may include one or more suitable salts and one or more suitable polyalkylene glycols, both of which are at respective concentrations suitable for binding polynucleotides to the binding surface, e.g., the surface of the magnetic microparticles. The kit further includes a wash solution comprising essentially, or exclusively, of an aqueous polyalkylene glycol solution for removing impurities from the binding surface while retaining most of the bound polynucleotides of interest. The wash solution removes impurities from the solid phase surface, but does not result in substantial elution of the polynucleotides bound to the anchoring surface, e.g., magnetic microparticles. Alternatively, instead of a wash solution, the kit can comprise the reagents for making the wash solution, to which a known amount of water can be added to create a wash solution at a preselected and desired concentration.

According to another aspect of the invention, a polynucleotides size selection kit is also provided which contains the reagents necessary for separating polynucleotides, such as DNA, RNA and PNAs, of desired lengths, from a solution containing a mixture of polynucleotides of different sizes. The kit includes a solid phase binding surface, e.g., magnetic microparticles with a carboxyl group-coated surface, and a binding buffer for facilitating reversible binding. The solid phase binding surface, in various embodiments, can be selected from: a microparticle, a beads, a membrane, a filter, a fiber, and a matrix. The binding buffer may include one or more suitable salts and one or more suitable polyalkylene glycols, both of which are at respective concentrations suitable for binding DNA of interested sizes to the binding surface, e.g., the surface of the magnetic microparticles. The kit further includes a wash solution comprising essentially, or exclusively, of an aqueous polyalkylene glycol solution for removing polynucleotides of unintended sizes from the binding surface while retaining most of the bound polynucleotide of interest. Alternatively, instead of a wash solution, the kit can comprise the reagents for making the wash solution, to which a known amount of water can be added to create a wash solution at a preselected and desired concentration. The kit may further include instructions on how to use the kit.

In one embodiment, the kit further includes an elution buffer that is capable of eluting or dissolving the polynucleotide, such as DNA or RNA, bound to the anchoring surface, e.g. magnetic microparticles. Alternatively, instead of a binding buffer, and/or elution buffer, the kit can comprise reagents for making the binding, and/or elution buffers, to which a known amount of water can be added to create binding, and/or elution buffers of desired concentrations.

In another embodiment, the kit includes reagents needed for clearing a cell lysate. In a preferred embodiment, the reagents are present in solutions at a concentration suitable for direct use in preparing a lysate without the need for further dilution.

The following examples are provided to illustrate the principles of the invention and are not meant to be limiting in any way.

### Example 1

### Wash Solution Removed Oligonucleotides and Retained DNA of Interest with Equivalent Efficacy as 70% Ethanol

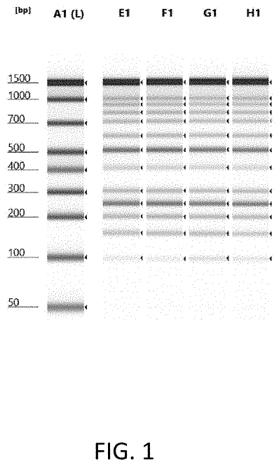

Referring to FIG. 1, in an exemplary embodiment, DNA of 50 bp or less in size was intended to be removed as impurities from DNA samples as follows:

Magnetic particles that have —COOH functional groups on their surface (commercially available from GE Healthcare) were prepared to 0.1% (w/v) in a solution of 50 mM Tris-HCl, pH 8.0. Separate wash solutions containing: (a) 30% (w/v) Polyethylene Glycol-2000 with 10 mM Magnesium Chloride, and (b) 70% ethanol were prepared.

1. 10 μL of a mixture of DNA fragments ranging from 50 base pair to 2000 base pair [thirteen individual DNA fragments of 1000, 900, 800, 700, 600, 500, 400, 300, 250, 200, 150, 100, 50 base pair in a solution containing 10 mM Tris-HCl (pH 7.6), 1 mM EDTA] were added into each well separately in a 96-well microtiter plate.

2. 18 μL of magnetic particle solution was added into each well containing DNA fragments, mixed and incubated at room temperature for 2 minutes.

3. The microtiter plate containing the samples was subsequently placed for 5 minutes in a magnetic holder.

4. The supernatant was discarded and the particles were washed twice with 200 μL of wash solution containing magnesium or 70% ethanol.

5. The microtiter plate was removed from the magnet and the particles were re-suspended in a 40 μL of elution solution (H₂O) immediately for samples washed with wash solution (a). Samples washed with 70% ethanol were air-dried for 5 minutes at room temperature before elution as standard practice.

6. The microtiter plate was placed in the magnet and the eluate was removed after 2 minutes.

Results: about 18 uL of purified DNA was loaded from each sample onto a 2200 TapeStation (Agilient). As shown in FIG. 1, wash solution (a) removed 50 bp DNA fragments completely and recovered similar concentrations of DNA of interest as 70% ethanol.

### Example 2

### Residual Wash Solution had no Negative Impact on PCR Reaction

Magnetic particles that have —COOH functional groups on their surface (commercially available from GE Healthcare) were prepared to 0.1% (w/v) in a solution of 50 mM Tris-HCl, pH 8.0. A wash solution containing 30% (w/v) Polyethylene Glycol-2000 with 10 mM magnesium chloride was prepared according to principles of the invention. Fresh 70% ethanol was prepared as control wash solution.

1. About 10 μL of Human DNA (10 ng/μL, commercially from Applied Biosystems, size from 10 bp up to 100,000 bp or more) was added to wells of a 96-well microtiter plate.

2. About 18 μL of magnetic particle solution was added into the each of the sample wells, mixed and incubated at room temperature for 2 minutes.

3. The microtiter plate containing the samples was subsequently placed for 5 minutes in a magnetic holder.

4. The supernatant was discarded and the particles were washed twice with 200 μL of the wash solution or 70% ethanol. At the end of each wash, wash solution or 70% ethanol was removed from the sample well and discarded.

5. The microtiter plate was removed from the magnet and the particles were re-suspended in a 40 μL of elution solution (H₂O) immediately for samples washed with wash solution of the invention. Samples washed with 70% ethanol were air-dried for 5 minutes at room temperature before elution as standard practice.

6. The microtiter plate was placed in the magnet holder and the eluate containing purified DNA was moved after 2 minutes into a clean vial.

7. Real-time PCR reaction setup: 4 μL of DNA purified with the wash solution containing Polyethylene Glycol was mixed in a well of a 96-well PCR plate with 5 μL of Power SYBR Green PCR Mastermix (2×, Applied Biosystems), 0.9 μL of forward PCR primer for β-actin gene (10 μM, Integrated DNA Technologies), and 0.9 μL of reverse PCR primer for β-actin gene (10 μM, Integrated DNA Technologies). 4 μL of DNA purified with 70% ethanol as wash solution was mixed in another well of the same PCR plate with the same set of reagents. The PCR plate was placed into a real-time PCR thermal cycler (CFX384 Real-Time System, BioRad), and went through a thermal cycling profile: Step 1: 95° C. for 10 minutes; Step 2: 95° C. for 15 seconds; Step 3: 60° C. for 1 minute. Step 2 and 3 were repeated for additional 39 cycles.

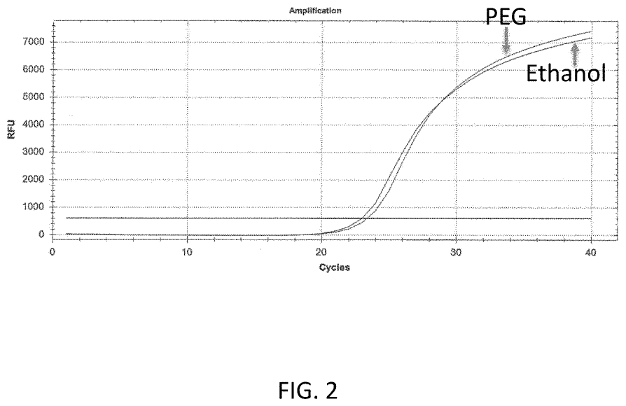

Results (FIG. 2): DNA purified with wash solution containing Polyethylene Glycol and Magnesium Chloride without an air-drying step (i.e. may carry over a small amount of residual wash solution) generated comparable amplification signals as DNA purified with 70% ethanol and processed with the air-drying step, indicating little or no impact from residual wash solution of the invention on downstream biochemical reactions, as well as a faster and more simplified workflow with the wash solution of the invention than with conventional ethanol-based wash solution.

### Example 3

### Wash Solution Successfully Removed Impurity that Inhibits Downstream PCR Reaction

Magnetic particles having —COOH functional groups on their surface (commercially available from GE Healthcare) were prepared in a solution of 50 mM Tris-HCl. A wash solution containing Polyethylene Glycol with Magnesium Chloride was prepared as in Example 2.

1. Human DNA (10 ng/μL, commercially from Applied Biosystems, size from 10 bp up to 100,000 bp or more) was spiked with 0.1M EDTA, a common impurity that inhibits enzymatic reactions such as polymerase chain reaction (PCR). Then, 10 μL of DNA/EDTA mixture was added to a well of a 96-well microtiter plate.

2. 18 μL of magnetic particle solution was added into the sample well, mixed and incubated at room temperature for 2 minutes.

3. The microtiter plate containing the samples was subsequently placed for 5 minutes in a magnetic holder.

4. The supernatant was discarded and the particles were washed twice with 200 μL of the wash solution. At the end of each wash, wash solution was removed from the sample well and discarded.

5. After the second wash the microtiter plate was removed from the magnet holder and the particles were re-suspended in a 40 μL of elution solution (H₂O).

6. The microtiter plate was placed in the magnet holder and the eluate containing purified DNA was moved after 2 minutes into a clean vial.

7. Real-time PCR reaction setup: 40 μL of purified DNA was mixed in a well of a 96-well PCR plate with 5 μL of Power SYBR Green PCR Mastermix (2×, Applied Biosystems), 0.9 μL of forward PCR primer for β-actin gene (10 μM, Integrated DNA Technologies), and 0.9 μL of reverse PCR primer for β-actin gene (10 μM, Integrated DNA Technologies). Then, 4 μL of un-purified DNA/EDTA was mixed in another well of the same PCR plate with the same set of reagents, as negative control. The PCR plate was placed into a real-time PCR thermal cycler (CFX384 Real-Time System, BioRad), and went through a thermal cycling profile: Step 1: 95° C. for 10 minutes; Step 2: 95° C. for 15 seconds; Step 3: 60° C. for 1 minute. Repeat step 2 and 3 for additional 39 cycles.

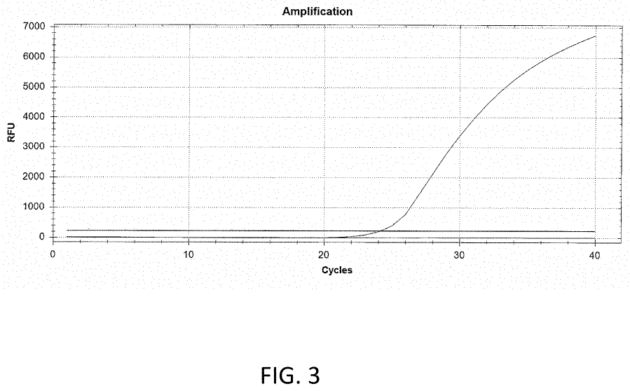

Results (FIG. 3): Un-purified DNA/EDTA solution failed to generate any amplification signal through PCR reaction. After purification with the wash solution containing Polyethylene Glycol, sample DNA generated significant amplification signal through PCR reaction.

Purification with wash solution containing Polyethylene Glycol successfully removed impurities in DNA solution. The purified DNA can be used for downstream enzymatic reactions such as PCR, free of negative or inhibitory impact from impurities.

### Example 4

### Wash Solution Improved Reproducibility of the Purification Result

Magnetic particles which have —COOH functional groups on their surface (GE Healthcare) were prepared in a solution of 50 mM Tris-HCl, pH 8.0. A wash solution containing Polyethylene Glycol with Magnesium Chloride and 70% ethanol control were prepared as in Example 1.

1. 10 μL of a mixture of DNA fragments ranging from 50 base pair to 1,000 base pair product [thirteen individual DNA fragments of 1000, 900, 800, 700, 600, 500, 400, 300, 250, 200, 150, 100, 50 base pair in a solution containing 10 mM Tris-HCl (pH 7.6), 1 mM EDTA] was added into each of six wells in a 96-well microtiter plate.

2. 18 μL of magnetic particle solution was added into each well containing DNA fragments, mixed and incubated at room temperature for 2 minutes.

3. The microtiter plate containing the samples was subsequently placed for 5 minutes in a magnetic holder.

4. The supernatant was discarded and the particles were washed twice with 200 μL of wash solution for three replicate wells or 70% ethanol for three replicate wells. At the end of each wash, wash solution was removed from the sample well and discarded.

5. The microtiter plate was removed from the magnet and the particles were re-suspended in a 40 μL of elution solution (H₂O) immediately for samples washed with wash solution. The samples washed with 70% ethanol were air-dried for 5 minutes at room temperature before elution as standard practice.

6. The microtiter plate was placed in the magnet holder and the eluate was removed after 2 minutes.

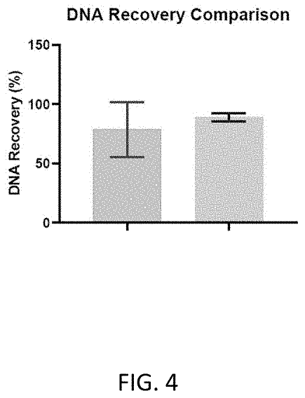

Results: In FIG. 4, concentrations of purified DNA with wash solution containing Polyethylene Glycol or 70% ethanol were assessed using a Nanodrop 2000 spectrometer (Thermo Scientific). Percentage of DNA recovery was calculated as recovered DNA amount divided by input DNA amount. Wash solution containing Polyethylene Glycol not only yielded higher amount of DNA than 70% ethanol, but also dramatically improved the reproducibility of the results between replicate samples.

### Example 5

### Purified RNA Subsequently Amplified

Magnetic particles that have —COOH functional groups were prepared as in Example 4. A wash solution according to an embodiment of the present invention containing Polyethylene Glycol with Magnesium Chloride was prepared. Fresh 70% ethanol was also prepared as control wash solution.

1. 10 μL of Human RNA (50 ng/μL, Affimatrix) was added to wells of a 96-well microtiter plate.

2. 18 μL of magnetic particle solution was added into the each of the sample wells, mixed and incubated at room temperature for 2 minutes.

3. The microtiter plate containing the samples was subsequently placed for 5 minutes in a magnetic holder.

4. The supernatant was discarded and the particles were washed twice with 200 of the wash solution of the invention or 70% ethanol. At the end of each wash, wash solution or 70% ethanol was removed from the sample well and discarded.

5. The microtiter plate was removed from the magnet and the particles were re-suspended in a 40 μL of elution solution (H₂O) immediately for samples washed with wash solution of the invention. Samples washed with 70% ethanol were air-dried for 5 minutes at room temperature before elution as standard practice.

6. The microtiter plate was placed in the magnet holder and the eluate containing purified DNA was moved after 2 minutes into a clean vial.

7. Reverse transcription reaction setup: 104, of purified RNA from the wash solution containing Polyethylene Glycol or 70% ethanol was mixed in a well of a 96-well PCR plate with qScript Reaction Mix (5×) 4μL, qScript Reverse Transcriptase 1 μL, water 5 μL. The PCR plate was placed into a real-time PCR thermal cycler (CFX384 Real-Time System, BioRad), and went through a thermal cycling profile: Step 1: 22° C. for 5 minutes; Step 2: 42° C. for 30 minutes; Step 3: 85° C. for 5 minutes.

8. Real-time PCR set up: 4 μL reverse transcription product from RNA purified with wash solution containing Polyethylene Glycol or 70% ethanol was mixed with 5 μL of Power SYBR Green PCR Mastermix (2×, Applied Biosystems), 0.94, of forward PCR primer for β-actin gene (10 μM, Integrated DNA Technologies), and 0.94, of reverse PCR primer for β-actin gene (10 μM, Integrated DNA Technologies). The PCR plate was placed into a real-time PCR thermal cycler (CFX384 Real-Time System, BioRad), and went through a thermal cycling profile: Step 1: 95° C. for 10 minutes; Step 2: 95° C. for 15 seconds; Step 3: 60° C. for 1 minutes. Steps 2 and 3 were repeated for additional 39 cycles.

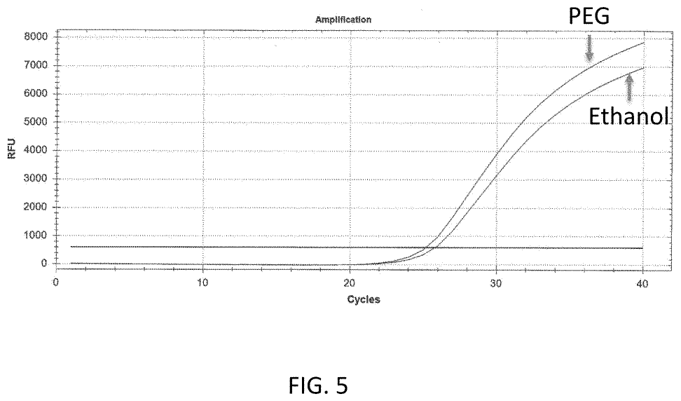

Results: RNA purified with wash solution containing Polyethylene Glycol according to an embodiment of the invention generated stronger amplification signal than RNA purified with 70% ethanol through reverse-transcription/Re al-time PCR (FIG. 5).

### Example 6

### Wash Solution with and without Magnesium Ions

Magnetic particles with —COOH functional groups were prepared as in Example 4. A wash solution containing Polyethylene Glycol with Magnesium Chloride and a same wash solution without Magnesium were prepared.

1. 10 μL of a mixture of DNA fragments ranging from 50 base pair to 1000 base pair product (thirteen individual DNA fragments of 1000, 900, 800, 700, 600, 500, 400, 300, 250, 200, 150, 100, 50 base pair in a solution containing 10 mM Tris-HCl (pH 7.6), 1 mM EDTA) were added into each well separately in a 96-well microtiter plate.

2. 18 μL of magnetic particle solution was added into each well containing DNA fragments, mixed and incubated at room temperature for 2 minutes.

3. The microtiter plate containing the samples was subsequently placed for 5 minutes in a magnetic holder.

4. The supernatant was discarded and the particles were washed twice with 200 μL of wash solution containing magnesium or wash solution without magnesium. At the end of each wash, wash solution was removed from the sample well and discarded.

5. After the second wash the microtiter plate was removed from the magnet holder and the particles were re-suspended in a 40 μL of elution solution (H₂O).

6. The microtiter plate was placed in the magnet holder and the eluate was removed after 2 minutes.

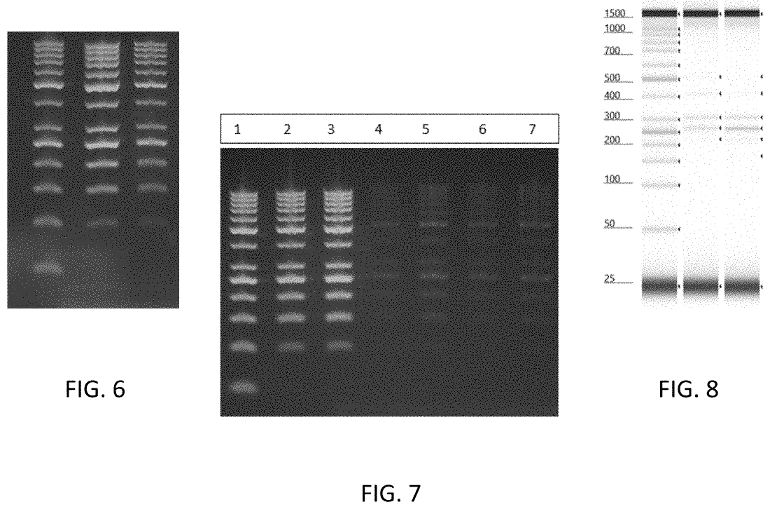

Results: Loaded 18 μL of purified DNA from each sample on a 2% agarose gel. As shown in FIG. 6, wash solution without magnesium yielded lower concentrations of DNA fragments of 100 base pair or larger than the wash solution with magnesium.

Accordingly, our data suggest that certain concentrations of salt, e.g. Magnesium Chloride, can be used as a component of a wash solution to purify or size-select polynucleotides, e.g., DNA.

### Example 7

### Wash Solution with Different Concentrations of PEGs of Various Molecular Weights

Magnetic particles with —COOH functional groups were prepared as in Example 4. Six different wash solutions were prepared: Buffer A contained 1% Polyethylene Glycol (v/v, molecular weight no more than 200, e.g., 200) with 10 mM Magnesium Chloride; Buffer B contained 1% Polyethylene Glycol (w/v, molecular weight between 200 and 1,000 inclusive both ends, e.g., 1,000) with 10 mM Magnesium Chloride; Buffer C contained 1% Polyethylene Glycol (w/v, molecular weight between 1,000 and 10,000 inclusive both ends, e.g, 10,000) with 10 mM Magnesium Chloride; Buffer D contained 1% Polyethylene Glycol (w/v, molecular weight higher than 10,000, e.g., 20,000) with 10 mM Magnesium Chloride; Buffer E contained Polyethylene Glycol at a concentration within 20-50% range (w/v) (e.g., 30%) with Magnesium Chloride at a concentration within 1-30 mM range (e.g., 10 mM); and Buffer F contained 70% ethanol.

1. 10 uL of a mixture of DNA fragments ranging from 50 base pair to 1000 base pair product (thirteen individual DNA fragments of 1000, 900, 800, 700, 600, 500, 400, 300, 250, 200, 150, 100, 50 base pair in a solution containing 10 mM Tris-HCl (pH 7.6), 1 mM EDTA) were added into each well separately in a 96-well microtiter plate.

2. 18 uL of magnetic particle solution was added into each well containing DNA fragments, mixed and incubated at room temperature for 2 minutes.

3. The microtiter plate containing the samples was subsequently placed for 5 minutes in a magnetic holder.

4. The supernatant was discarded and the particles were washed twice with 200 μL of each of the six types s. At the end of each wash, wash solution was removed from the sample well and discarded.

5. After the second wash the microtiter plate was removed from the magnet holder and the particles were re-suspended in a 40 μL of elution solution (H₂O).

6. The microtiter plate was placed in the magnet holder and the eluate was removed after 2 minutes.

Results: Loaded 18 uL of purified DNA from each sample on a 2% agarose gel. As shown in FIG. 7, wash solutions with Polyethylene Glycol molecular weight 100 to 20,000 at as low as 1% concentration (w/v) were able to retain some DNA fragments of 100 base pair or larger, even though the concentration of DNA retained is not as high as a more optimal formulation such as 20-50% Polyethylene Glycol (w/v) with Magnesium Chloride of 1-30 mM (Lane 3) or the prior art (70% ethanol, Lane 2).

### Example 8

### Wash Solution Removed DNA of Unintended Sizes and Retained DNA of Intended Sizes with Improved Efficacy Compared to 80% Ethanol

Referring to FIG. 8, in an exemplary embodiment, 200-300 bp DNA was intended for sequencing while DNA of smaller and larger sizes outside the range was to be removed through a size-selection process as follows:

Magnetic particles that have —COOH functional groups on their surface (commercially available from GE Healthcare) were prepared in a solution of 50 mM Tris-HCl, pH 8.0. Separate wash solutions containing either Polyethylene Glycol with Magnesium Chloride or 70% ethanol were prepared.

1. 40 μL of a mixture of DNA fragments ranging from 50 bp to 2000 bp product were added into each well separately in a 96-well microtiter plate.

2. 28 μL of magnetic particle solution was added into each well containing DNA fragments, mixed and incubated at room temperature for 2 minutes.

3. The microtiter plate containing the samples was subsequently placed for 5 minutes in a magnetic holder.

4. For each sample the supernatant was transferred to a new well and the particles were discarded.

5. 6 μL of magnetic particle solution was added into each well containing transferred supernatant, mixed and incubated at room temperature for 2 minutes.

6. The microtiter plate containing the samples was subsequently placed for 5 minutes in a magnetic holder.

7. The supernatant was discarded and the particles were washed twice with 200 μL of wash solution or ethanol. At the end of each wash, wash solution was removed from the sample well and discarded.

8. After the second wash the microtiter plate was removed from the magnet holder, and the particles were re-suspended in a 20 μL of elution solution (H₂O) immediately for samples washed with wash solution. Samples washed with 70% ethanol were air-dried for 5 minutes at room temperature before elution as standard practice.

9. The microtiter plate was placed in the magnet and the eluate was removed after 2 minutes.

Results: about 5 uL of eluted DNA was loaded for each sample on a TapeStation 2200 (Agilent). As shown in FIG. 8, wash solutions with Polyethylene Glycol selectively retained DNA of 200-300 bp as intended sizes, while removed larger and smaller DNA fragments outside the intended size range. 80% ethanol selectively retained DNA of the same range, but with more residual larger DNA and lower concentration of DNA of intended sizes.

Our data suggests that Polyethylene Glycol of molecular weight 100 up to 20,000 at as low as 1% concentration, preferably at about 10-70% inclusive both ends, more preferably 20-50% inclusive both ends (e.g., about 30%) can be used as wash solution to purify DNA and other polynucleotides.

While the present invention has been particularly shown and described with reference to the structure and methods disclosed herein and as illustrated in the drawings, it is not confined to the details set forth and this invention is intended to cover any modifications and changes as may come within the scope and spirit of the following claims. All publications and patent literature described herein are incorporated by reference in entirety to the extent permitted by applicable laws and regulations.

What is claimed is:

1. A method of purifying polynucleotides without use of any low-weight alcohol, the method comprising the sequential steps of:
(a) contacting a solid surface with a solution containing polynucleotides of interest and impurities;
(b) allowing binding between the surface and the polynucleotides of interest to take place; and
(c) washing the polynucleotide-bound surface with a wash solution consisting essentially of polyalkylene glycol and water to remove impurities from the surface.

2. The method of claim 1, further comprising the following step after step (c):
(d) eluting the polynucleotides from the surface.

3. The method of claim 1, wherein step (a) comprises contacting the solid surface with a binding buffer comprising polyalkylene glycol.

4. The method of claim 1, wherein the solid surface is selected from a group consisting of a microparticle, a beads, a membrane, a filter, a fiber, and a matrix.

5. The method of claim 1 wherein the polyalkylene glycol ranges from 1% to about 100% in the wash solution.

6. The method of claim 1 wherein the polyalkylene glycol comprises polyethylene glycol.

7. The method of claim 6, wherein the molecular weight of polyethylene glycol is between 100 to 20,000, inclusive of both ends.

8. The method of claim 6, wherein the molecular weight of polyethylene glycol is between 200 to 10,000, inclusive of both ends.

9. The method of claim 6, wherein the wash solution is about 20%-50% (w/v) in polyethylene glycol.

10. The method of claim 1 wherein the polyalkylene glycol comprises polypropylene glycol.

11. The method of claim 1, wherein the polynucleotides are selected from a group consisting of DNAs, RNAs, and PNAs.

12. The method of claim 1, wherein the wash solution also has 1-30 mM ionic salt.

13. A method of selecting polynucleotides of a desired size from a mixture of polynucleotides without use of any low-weight alcohol, the method comprising the sequential steps of:
(a) contacting a solid surface with a solution containing polynucleotides of different sizes comprising a desired size and an unintended size;
(b) allowing binding between the surface and the polynucleotides to take place; and
(c) washing the polynucleotide-bound surface with a wash solution consisting essentially of polyalkylene glycol and water to remove an amount of polynucleotides of the unintended size from the surface.

14. A polynucleotide purification or size selection kit free of low-weight alcohol, the kit comprising:
a solid phase binding surface and a binding buffer for facilitating reversibly binding a polynucleotide of interest in a solution to the solid phase binding surface; and
a wash solution consisting essentially of polyalkylene glycol and water for removing impurities from the binding surface while retaining most of the bound polynucleotides of interest.

15. The kit of claim 14, wherein the solid phase binding surface is selected from a group consisting of a microparticle, a beads, a membrane, a filter, a fiber, a matrix.

16. The kit of claim 14, wherein the polyalkylene glycol ranges from 1% to about 100% in the wash solution.

17. The kit of claim 14, wherein the polyalkylene glycol comprises polyethylene glycol.

18. The kit of claim 14, wherein the molecular weight of polyethylene glycol is between 100 and 20,000, inclusive of both ends.

19. The kit of claim 14, wherein the wash solution is about 20%-50% in polyethylene glycol.

20. The kit of claim 14 wherein the polyalkylene glycol comprises polypropylene glycol.
